(19) United States
(12) Patent Application Publication
(10) Pub. No.: US 2015/0190461 A1
(43) Pub. Date: Jul. 9, 2015

(54) DOSING INSTRUCTIONS FOR ENDOTOXIN-BINDING LIPOPEPTIDES

(71) Applicant: Zentrum für Biomedizinische Technologie der Donau- Universität Krems, Krems (AT)

(72) Inventors: Dieter Falkenhagen, Krems (AT); Stephan Harm, Durnstein (AT); Jens Hartmann, Furth (AT)

(21) Appl. No.: 14/411,879

(22) PCT Filed: Jun. 23, 2013
(86) PCT No.: PCT/EP2013/063496

§ 371 Date: Mar. 19, 2015

(30) Foreign Application Priority Data
Jun. 29, 2012 (EP) 12174285.2

(51) Int. Cl. A61K 38/12; A61K 9/00

(52) U.S. Cl. CPC A61K 38/12; A61K 9/0019

(57) ABSTRACT

The invention relates to an endotoxin-binding lipopeptide selected from the group consisting of polymyxins, polymyxin derivatives, polymyxin analogues, prodrugs thereof and pharmaceutically acceptable salts thereof, and also relates to a preparation for parenteral administration comprising a lipopeptide of this type, for prophylaxis or for treatment of diseases and conditions caused by endotoxemia, by i) parenterally administering a bolus of the lipopeptide to achieve a lipopeptide serum concentration of 0.01 μg/ml to 0.8 μg/ml and ii) maintaining this lipopeptide serum concentration by parenterally administering the lipopeptide over a specifiable period of time.

The invention relates to an endotoxin-binding lipopeptide selected from the group consisting of polymyxins, polymyxin derivatives, polymyxin analogs, their prodrugs, and pharmaceutically acceptable salts thereof for prophylaxis or treatment of diseases and conditions caused by an endotoxin. The invention also concerns a method for parenteral administration, comprising at least one such endotoxin-binding lipopeptide as an active ingredient and a pharmaceutically acceptable carrier and/or excipient for prophylaxis or treatment of diseases and conditions caused by endotoxemia.

Endotoxins are lipopolysaccharides (LPSs) in the cell wall of Gram-negative bacteria, and they are released by cell lysis and cell splitting. In fact, lipopolysaccharides are the most frequent lipid component of the outer cell membrane of Gram-negative bacteria. Endotoxins are pyrogenic substances, and affected individuals react with a strong inflammation reaction and fever when endotoxins, for example, in consequence of a microbial poisoning, enter the body and act as key mediators of an uncontrolled activation of the mononuclear phagocyte system. An accumulation of endotoxins in the blood circulation in consequence of endotoxemia leads to uncontrolled activation of the immune cells and an imbalance of the clotting system. This can lead to sepsis characterized by, among other things, high fever, low blood pressure, and in serious case, multiple organ failures. Sepsis is a disease that must be taken very seriously; the lethality of individuals with severe sepsis is about 30-60%, depending on the degree of severity. Endotoxemia due to an infection with Gram-negative bacteria is one of the most frequent causes of the appearance of a systemic inflammatory response (systemic inflammatory response syndrome, SIRS), serious sepsis, or septic shock and serious complications resulting therefrom. Patients with compromised immune defenses, such as, e.g., liver patients or patients in chemotherapy, have tendency to bacterial infection and thereby show symptoms of endotoxin poisoning. Endotoxemia can likewise appear in cases of acute liver failure or acute decompensation in cases of chronic liver failure, through which conditions [arise] that—viewed biochemically—are very similar to sepsis. For example, in patients with chronic liver failure, an acute decompensation can arise. In this conditions, endotoxins originating from normal intestinal flora can overcome the intestinal barriers and stimulate the release of inflammation mediators in the body, thus causing a condition similar to sepsis.

Lipopolysaccharide molecules have a three-part structure: a lipid A forms the region of the molecule that faces the bacterial cell; through the lipid A, the molecule; the molecule is anchored to the outer membrane of a Gram-negative bacterium. The LPS molecule also has a middle core region connected to the lipid A that is highly conservative. The third and outermost region consists of an O-specific polysaccharide (O-antigen), the structure of which can vary strongly among the various Gram-negative bacteria. The toxic effect derives from the lipid A, which is only released during cell lysis.

Polymyxins are antibiotic substances that originally derived from the bacterium Bacillus polymyxa and have been used for a long time in the treatment of infections with Gram-negative bacteria in humans and animals. Polymyxins reach into the cell-wall structure, in which they increase the permeability of the cell membrane, because of which cell lysis occurs. Polymyxins bind not only to phospholipids, but also to lipopolysaccharides (endotoxins) with high affinity. The antibacterial mechanism of polymyxins is described thoroughly, for example, in a publication by Tony Velkov et al. (Tony Velkov et al. 2010. Journal of Medicinal Chemistry: 53 (5): 1898-1916).

Because of the neurotoxic and nephrotoxic effect of polymyxins, only polymyxin B and polymyxin E (colistin) have achieved a certain therapeutic importance as antibiotics. Up to this point in time, only polymyxin B and are approved by the FDA in the USA for parenteral infusion. Polymyxin B and have been approved for decades for oral or topical forms of therapy. For parenteral systemic treatment in cases of diseases and conditions due to an infection with Gram-negative bacteria, however, due to their neuro- and nephrotoxic side effects, they are used therapeutically only as the last possible solution. Colistin seems to be less nephrotoxic than polymyxin B, but this is partly offset by the higher dosage required, so that in everyday clinical practice, nephrotoxic reactions can be expected to the same extent. Sufficient data on the nephrotoxicity of both these antibiotics does not exist at this time, however. Infectologists from New York (USA) describe kidney failure in 14% of 60 patients who were treated with polymyxin B. Physicians in Greece describe clear nephrotoxicity in most of the patients in whom renal insufficiency existed already at the start time the therapy was started. In contrast, however, in patients with normal kidney function, no essential changes were observed. A detailed overview of the toxicity of polymyxins is round in a publication by Falagas and Kasiakou (Falagas and Kasiakou, 2006, Critical Care 10:R27). The dosing of polymyxins consequently plays a central role in avoiding or minimizing toxic side effects, especially nephrotoxic side effects.

Because of the increase observed in the appearance or serious disease coursed due to acute infections and multiresistant pathogenic strains, for example in acute infections and strains of the bacterium Pseudomonas aeruginosa, polymyxins have been applied parenterally as an antibiotic in spite of their toxicity. A reference source for polymyxin B in the form of the sulfate salt of polymyxins B1 and B2 for parenteral administration has recently been offered by Bedford Laboratories (“Polymyxin B for Injection, 500,000 units,” manufacturer: Bedford Laboratories). According to the manufacturer's information, parenteral administration is done intravenously, intramuscularly, or in the case of meningitis, intrathecally, whereby the maximum daily dosage is, as a rule, 2.5 mg/kg of body weight, divided into two or three infusions. Typically, the serum concentration of polymyxin after administration is in a range from 1 to 6 μg/ml. In serious cases, this can also be higher, in a range from 6 to 50 μg/ml. (polymyxin E) is used in a manner similar to polymyxin B, at most with the difference of a higher dosage. Resistance to polymyxin B is fairly unusual, but it can develop when the antibiotic does not reach the cytoplasm membrane due to changes in the outer membrane. Polymyxins are effective against many Gram-negative pathogens such as, e.g., E. Coli, Enterobacter, and Klebsiella spp., and also against P. aeruginosa. Proteus types, and S. marcescens, which are normally resistant; the sensitivity of B. fragilis is variable. The minimal inhibitory concentrations for E. coli are in the range from 0.04 to 3.7 mg/l and for P. aeruginosa between 1.2 and 33.3 mg/l (Garidel and Brandenburg, 2009, Anti-Infective Agents in Medicinal Chemistry, 8:367-385).

Since the dosages used so far for polymyxin B and induce nephro- and neurotoxic effects with parenteral administration, new treatment strategies and therapeutic approaches were developed in the past in combination with the use of endotoxin-binding lipopeptides such as polymyxin.

As a frequently applied alternative to the application of polymyxins in the form of a medication, extracorporeal blood and/or blood-plasma cleaning methods (therapeutic apheresis) have been established using suitable adsorption materials. Apheresis methods and adsorbing materials to eliminate toxic and/or damaging substances from blood and blood plasma are well known in the state of the art. Known adsorbing materials include porous or fiber-like carrier materials, on the surfaces of which polymyxin B, for example is immobilized covalently or by means of hydrophobic interaction. A apheresis gene has already been reported in connection with such adsorbing materials that is highly in the treatment of septic conditions, has no neuro- and nephrotoxic side effects. Adsorbing materials that are functionalized with polymyxin B are known, for example, from EP 0,110,409 A1, WO 2010/083545, and WO 2011/160149. Apheresis methods using suitable adsorbers have, however, the disadvantage that, because of the high technical cost, limited availability of therapy sites, and essentially higher manufacturing and therapy costs compared to treatment with medications, they cannot be used extensively, and therefore only needs for intensive medical care can be covered.

Although the lethality of patients with endotoxin-induced diseases, especially sepsis could be reduced with the use of the above-mentioned polymyxin-based adsorbing materials, the lethality of patients with severe sepsis and septic shock is still very high, in spite of maximal therapy. Multiresistance of bacteria to antibiotics and the associated increasing incidence of severe disease courses, and an extensive need still exists for cost-favorable forms of therapy with low side effects. Consequently, in recent years, therapeutic approaches with medicines have been used again. For example, through chemical modification of the Dab side chains, the cyclic peptide ring, or the fatty-acid chains of the polymyxin molecule structure, a number of synthetic polymyxin analogs/derivatives have been developed in order to create a number of medications with low toxic side effects and/or improved endotoxin efficiency. However, most of these modifications have led to inactive compounds. Furthermore, no toxicological data are known for many of the analogs/derivatives that have been described. A detailed overview of polymyxin-based antibiotics, analogs, and derivatives is given in the publication by Velkov et al. (Velkov et al., 2010, Journal of Medicinal Chemistry, 53 (5): 1898-1916).

EP 2,332,965 describes synthetic peptides derived from naturally occurring polymyxins and octapeptides with antibacterial properties for use as antibiotics to treat individuals with a bacterial infection, as well as a method for producing these peptides, especially in the form chemical compounds derived from polymyxin B that have antibacterial properties as antibiotics against a number of Gram-negative bacteria. The compounds described therein have reduced toxicity compared to polymyxin B.

WO 2010/075416 discloses polymyxins, especially chemical compounds derived from polymyxin B with antibacterial properties as antibiotics against a variety of Gram-negative bacteria. The compounds described therein have reduced toxicity compared to polymyxin B.

In spite of the numerous approaches to the manufacture of peptide compounds to create a medication endotoxin-binding efficiency and/or antibacterial effect comparable to that of polymyxin B or and with clearly reduced toxicity, none of these compounds as been approved for clinical use.

It is therefore a task of the invention to eliminate or minimize the known disadvantages of the state of the art and provide new dosing instructions for medicinally applied endotoxin-binding lipopeptides such as polymyxins, polymyxin analogs, or polymyxin prodrugs and pharmaceutically acceptable salts thereof, so that prophylaxis or treatment of illnesses and conditions that are caused by endotoxemia is made possible. It is especially a task of the invention to provide new dosing instructions for clinical use of permitted polymyxins such as polymyxin B and. The new dosing instructions should bring with it significant improvements in regard to toxic side effects with unchanged high effectiveness and essentially represent a more cost-favorable therapy possibility for patients with endotoxemia than the adsorbing materials used so far for these purposes.

This task is solved through new dosing instructions characterized by i) parenteral application of a bolus of lipopeptides to achieve a lipopeptide to achieve a lipopeptide serum concentration from 0.1 μg/ml to 0.8 μg/ml and

ii) maintaining this lipopeptide serum concentration through parenteral application of lipopeptides over a specifiable period of time.

Thanks to the invention, it is possible for the first time to use polymyxins and/or polymyxin derivatives and polymyxin analogs not in the traditional sense as antibiotics, but to eliminate Gram-negative bacteria in a clearly lower concentration (4 to 100 times lower) to inactivate endotoxins in patients with endotoxemia. Inactivation of endotoxins means that their biological effect, especially on the release of inflammation mediators like cytokines is inhibited or blocked. This has as a consequence that the proinflammatory phase, such as that caused in sepsis or in SIRS, is suppressed or reduced.

The new kind of dosing instructions make parental administration of polymyxins and their analogs, derivatives, and prodrugs possible, whereby nephro- and neurotoxic side effects can be avoided. The lipopeptide serum concentration when the dosing instructions according to the invention is used below the concentration achieved by using the permitted polymyxin B preparations for parenteral application by a factor 4 to 100.

The surprising factual situation has been found that already very low serum concentrations of polymyxin B from 0.01 μg/ml to 0.8 μg/ml are sufficient to inhibit the endotoxins in their activity, whereby neuro- and nephrotoxic effects are excluded. The effect of these new types of dosing instructions has been demonstrated on endotoxins from E. coli and Pseudomonas aeruginosa, whereby a starting endotoxin concentration of 0.5 ng/ml was selected, thus a concentration that already represents a maximum value in a clinically relevant sepsis.

The dosing instructions according to the invention are based on the surprising factual situation that during the study, various adsorbing materials functionalized with polymyxin desorbed amount an endotoxin binding exclusively in a very small amount and polymyxin molecules were subsequently transferred got into the blood or blood plasma. These astonishing and unforeseeable results are based on the factual situation that after targeted washing of the adsorbing materials, during which desorbable polymyxin molecules are removed from absorbing surface, no endotoxin adsorption to the polymyxin materials still immobilized adsorbing materials could be detected. It was established that also with a covalent binding of polymyxin to the adsorbing surface, a certain amount of polymyxin molecules were bound by unspecified forces. The non-specifically bound polymyxin molecules can be desorbed from the adsorbing surfaces during therapeutic use and in the free state make endotoxins in the blood or blood plasma of patients non-damaging. It can thus be established in summary, that the very good endotoxin adsorption by adsorbing materials based on porous or fibrous carrier materials on the surface of which polymyxin B is immobilized, for example covalently or by means of hydrophobic interaction, can be attributed exclusively to a very small amount of polymyxin molecules that released into the blood or blood plasma. The binding between polymyxin and endotoxin obviously takes place only when both the hydrophobic and the positively charged amino groups of polymyxin B are accessible to the endotoxins.

The new type of dosing instructions as described in this disclosure is based on these surprising results, and it defines essentially lower serum concentration of endotoxin-binding lipopeptides such as polymyxin compared to those have been established as standard therapies that have been performed for many decades with polymyxin B and colistin.

The concepts “polymyxin” and “polymyxins” as used here relate to known naturally occurring chemical compounds that derived originally from the bacterium Bacillus polymyxa (polymyxin B) and from Bacillus colistinus (polymyxin E).

Polymyxins can be isolated either from bacteria or produced synthetically. The polymyxins B deriving from bacteria consist of 6 derivatives, which are called polymyxin B1, polymyxin B2, polymyxin B3, polymyxin B4, polymyxin B5, and polymyxin B6. In contrast to this, the polymyxin approved by the FDA for parenteral infusion is composed only of polymyxin B1 through B4.

The concept “polymyxin derivative” relates to a compound derived from one of the polymyxins that can be obtained through modification of naturally occurring polymyxins, for example by chemical modification of the Dab side chain, the cyclic peptide ring, or the fatty-acid chain of the polymyxin molecule structure. A detailed overview of polymyxin-based antibiotics, analogs, and derivatives in described in the publication by Velkov et al. (Velkov et al., 2010, Journal of Medicinal Chemistry, 53(5):1898-1916). A representative example of a polymyxin derivative is polymyxin nonapeptide, a derivative of polymyxin B that lacks the hydrophobic part and one amino acid.

The concept “polymyxin analog” as used herein relates to a chemical lipopeptide compound that is structurally similar or comparable to a polymyxin (“polymyxin-like lipopeptide”) and has the same endotoxin-binding effect as polymyxins or an endotoxin-binding effect comparable to one with polymyxins. A representative example of such a polymyxin analog can be seen in the disclosure WO 2008/006125 A1.

The concept “prodrug” as used herein relates to a preliminary compound of the endotoxin-binding lipopeptide as defined, whereby the preliminary compound is converted in vivo into the active endotoxin-binding lipopeptide. As representative examples, the prodrugs colistin methane sulfonate and polymyxin B methane sodium can be mentioned.

The concept “endotoxemia” is used herein for all disease causers in which clinically relevant amounts of endotoxins can be founts in the blood of patients that subsequently lead to the induction of cytokines, advantageously in disease pictures such as sepsis and SIRS.

The dosing instructions are suitable for both treatment and prophylaxis of diseases and conditions that are caused by endotoxemia. By “prophylaxis,” an application of the endotoxin-binding lipopeptide is to be understood when endotoxemia is present, but no clinical symptoms are present. Prophylactic therapy can be indicated especially in patients in whom endotoxemia is to be considered on the basis of their disease, for example in patients suffering from acute liver failure or an acute decompensation in chronic liver failure, so that the dosing instructions according to the invention can be applied with the appearance of endotoxemia corresponding to the endotoxin-binding lipopeptides already before the appearance of clinical symptoms.

In addition, it is known that when antibiotics are used in an infection with Gram-negative bacteria and through the cell lysis induced by administering the antibiotics, an increased endotoxin release comes or can come. The invention can therefore have an advantage as an additional therapeutic or prophylactic step with the framework of conventional treatment of bacterially induced diseases by release of endotoxins that lead to the induction of cytokines in order to capture the endotoxins release due to the induction of cytokines.

The concept “parenteral administration” as used herein relates to administration other than enteral and topical administration, especially to an injection or infusion, whereby the injection or infusion can preferably take place intravenously, subcutaneously, intramuscularly, intra-arterially, or intrathecally, without being limited to these. Parenteral administration has the advantage that the serum lipopeptide concentration to be reached can be set quickly and maintained both when giving the initial bolus and when maintaining it over a specifiable period of time. Parenteral administration takes place advantageously in the form of an intravenous infusion or in an extracorporeal blood circulation as described in detail below.

The invention can be used both the human and veterinary medical fields. The concept “patient” as used herein consequently relates to both humans and animals. Because of the increased appearance of infections by multiresistant strains and the associated need for new forms of therapy, the invention has high relevance especially for the field of human medicine.

Since the naturally occurring polymyxins that come initially from the bacterium Bacillus polymyxa are some of the most studied peptide antibiotics and have been used for decades in the treatment of diseases and conditions due to endotoxemia, it is preferred that the endotoxin-binding lipopeptide by a polymyxin. It is especially preferred that the lipopeptide be selected from the group consisting of the polymyxins polymyxin B and colistin (polymyxin E), which are so far the only polymyxins that have been approved for clinical use. Polymyxin B is most preferred, however, since it has turned out to be the best for use in the field of human medicine. Polymyxin B is used preferably in the form of polymyxin-B sulfate.

In addition, another object of the invention is a preparation for parenteral administration comprising at least one endotoxin-binding lipopeptide as defined in this disclosure as an effective ingredient and optionally a pharmaceutically acceptable carrier and/or excipient. The preparation can be only one type of endotoxin-binding peptide or it can consist of a mixture of two or more endotoxin-binding lipopeptides, for example a mixture of polymyxins B1, B2, B3, and B4.

A “pharmaceutically acceptable carrier or excipient” can be any substance that is known for the production of parenteral application forms such as injections, infusion solutions, etc. Formulations of injection and infusion solutions that are suitable for the invention are listed below in example 5.

The preparation for parenteral administration is preferably in the form of an injection preparation or infusion preparation. The endotoxin-binding lipopeptide is preferably in the form of a freeze-dried powder for production of a sterile aqueous injection preparation or infusion preparation, whereby the powder can be dissolved in, for example, a 5% dextrose solution in sterile water, a Ringer solution, or a physiological sodium-chloride solution.

The lipopeptide in the preparation is present in dissolved form in step i) for parenteral administration of a bolus, preferably in a concentration of 5 mg/l to 200 mg/l, and in step ii) for maintaining the serum concentration it is preferably present in a concentration from 0.04 mg/l to 13 mg/l, more preferably from 0.1 mg/l to 7 mg/l, and most preferably from 0.5 mg/l to 4 mg/l.

The serum lipopeptide concentration is preferably in a range from 0.1 μg/ml to 0.6 μg/ml, more preferably 0.1 μg/ml to 0.4 μg/ml, most preferably between 0.1 μg/ml to 0.25 μg/ml, since at these serum concentrations, even in serious disease courses such as sepsis, severe sepsis, or septic shock, an efficient therapy can be performed without neuro- and nephrotoxic side effects.

The serum lipopeptide concentration according to the invention as defined in a first step i) as defined in the claims can be achieved through giving a one-time bolus of the lipopeptide; in order to maintain the desired serum concentration. Within the framework of this disclosure, by “bolus” is meant a one-time parenteral administration of the endotoxin-binding lipopeptide in the form of a preparation, preferably in the form of an injection or infusion preparation, whereby the preparation for giving the bolus preferably has a higher concentration of the lipopeptide than the preparation that is used in step ii) to maintain the serum lipopeptide concentration.

The bolus in step i) is preferably given over a period of at least 10 minutes, more preferably at least 60 minutes, and most preferably over at least 120 minutes, advantageously through a parenteral injection or infusion, ideally not during a dialysis treatment. To determine the amount of polymyxin required, the patient's blood volume is to be taken into account. The bolus (step i)) is preferably given as an injection, whereby, for example, a one-time bolus of 10 to 250 ml of the prepared injection solution is applied at the start of the treatment. The bolus can also be administered by means of an infusion.

In a second step ii), the serum lipopeptide concentration set rapidly by means of giving the bolus is maintained over a specifiable period of time, whereby the serum concentration is maintained advantageously by giving an infusion that is performed continuously. The infusion rate depends on the serum half-life of the lipopeptide in the patient. For example, the serum half-life for polymyxin B in patients with normal kidney function is typically 13 hours, for colistin 6 to 7.4 hours, according to the information in the literature. With simultaneous treatment by means of an extracorporeal blood-cleaning method as described below, the clearance of the filter and/or the clearance of an adsorbing system for the lipopeptide is also to be taken into account.

The time period for maintaining the serum lipopeptide concentration in step ii) is preferably the entire time period of the therapy, thus as long as an endotoxemia exists. This time period can be from a few hours to 2 weeks or even longer, whereby during this time period, the undesired induction of cytokines is reduced or suppressed.

A detailed description of how the desired serum concentration can be reached is found below in the examples.

In a variant of the invention, which can be used especially cost-favorably and extensively, the serum lipopeptide concentration is maintained by intravenous administration. In this variant, the endotoxin-binding lipopeptide is administered intravenously, preferably by means of a vein access, advantageously by means of a dosing pump.

In another variant, the serum lipopeptide concentration is maintained in step ii) through an extracorporeal perfusion system by infusion into blood of a patient in an extracorporeal blood circulation at a position upstream from a dialyzer (dialysis filter). This variant is indicated as an additional therapeutic step especially in serious or life-threatening patient conditions (e.g., sepsis) that make intensive medical steps in the form of extracorporeal blood and/or plasma cleaning (therapeutic apheresis) required. The lipopeptide is infused through a line into the blood circulating in the extracorporeal blood circulation at a position upstream from the dialyzer, thus directly before the blood is returned to the patient. Although direct intravenous administration is preferred for giving the bolus in step i), the bolus can also be injected into the extracorporeal blood circulation through a line upstream from the dialyzer. The bolus should be administered preferably at a time before the start of dialysis treatment; the continuous infusion at the same time as the dialysis, at a position preferably upstream from the dialyzer.

Since in this variant the endotoxin-binding lipopeptide is broken down not only by the body, but also removed from the blood by the dialyzer, it is appropriate to take the lipopeptide clearance by the body and the lipopeptide clearance of the dialyzer into account.

If an enrichment device is also arranged in the extracorporeal perfusion system, for example in the form an adsorbing cartridge or a plasma circulation with adsorbing particles suspended therein, it is favorable if the lipopeptide clearance of an enrichment device arranged in the extracorporeal perfusion system is also taken into account in dosing the infused lipopeptide. For example, it is known that adsorbers of a polystyrene/divinyl-benzene copolymer also adsorb lipopeptides like polymyxins in addition to pathophysiologically relevant components such as cytokines, so that taking the lipopeptide clearance of the adsorber into account is advantageous for dosing the infused lipopeptides.

The invention is used advantageously to treat an infection with Gram-negative bacteria, especially for prophylaxis or treatment of a systemic inflammatory reaction (SIRS), sepsis, serious sepsis, or septic shock. Representative examples of disease pictures that can be treated according to the present disclosure are those that can appear due to an infection with Gram-negative bacteria and can develop subsequently into SIRS, sepsis, serious sepsis with multiple organ failure, or septic shock.

Representative examples of Gram-negative bacteria are Escherichia spp., Haemophilus influenzae, Pseudomonas aeruginosa, Pasteurella, Enterobacter spp., Salmonella spp., and Shigella spp. The invention is especially advantageous in Gram-negative bacteria for which an increased appearance of multiresistant strains is observed, whereby Pseudomonas aeruginosa is mentioned here as an especially relevant representative.

As already mentioned above, when antibiotics are used in an infection with Gram-negative bacteria, an increased release of endotoxins comes or can come through the cell lysis induced by the administration of antibiotics. An increased release of endotoxins has been described, for example for antibiotics that bind preferably to the PBP-3 (“penicillin-binding protein-3”), e.g., the frequently used antibiotics of the group of cephalosporins such as ceftazidimin. The invention can therefore be used advantageously in addition to therapeutic or prophylactic steps within the framework of a conventional treatment of bacterial infectious diseases by means of antibiotics to capture endotoxins released by the induction of cytokines.

In an additional aspect, the invention can be used advantageously for prophylaxis or treatment of an inflammation reaction in consequence of an acute liver failure or an acute decompensation in case of chronic liver failure, especially a systemic inflammatory reaction (SIRS), sepsis, severe sepsis with multiple organ failure, or septic shock. In patients with intact liver function, the endotoxins appearing in the blood circulation from the reticuloendothelial system (RES) or stellate Kupffer cells are eliminated through endocytosis. In patients with chronic liver failure, acute decompensation can come. In this case, endotoxins or normal intestinal flora can overcome the intestinal barrier and thereby pass the liver unhindered and lead to a systemic inflammatory reaction (SIRS), sepsis, severe sepsis with multiple organ failure, or septic shock.

The invention also concerns a method for prophylaxis or treatment of diseases and condition that are caused by endotoxemia, by application of an endotoxin-binding lipopeptide selected from the group consisting of polymyxins, polymyxin derivatives, polymyxin analogs, and prodrugs and pharmaceutically acceptable salts thereof, through i) parenteral administration of a bolus of the lipopeptide to achieve a serum lipopeptide concentration of 0.01 μg/ml to 0.8 μg/ml and ii) maintaining the serum lipopeptide concentration through parenteral application of the lipopeptide over a specifiable period of time. The above-mentioned definitions and further developments are to be used equally for the method.

The invention will be described in more detail in the following by means of non-limiting examples.

### EXAMPLE 1

### Endotoxin (LPS) Inactivation Depending on the Polymyxin Concentration by Means of Endotoxins from E. coli and Pseudomonas aeruginosa

1.1. Goal

The goal of this experiment is to determine the inactivation of endotoxins depending on the concentration of polymyxin B (PMG) in the plasma (batch test I). Also to be studied is to what extent this endotoxin elimination has the consequence of inhibiting the release of cytokines (batch test II).

1.2. Blood Donation

A blood donation was taken in 9 blood-donation tubes (9 ml each) and spiked with 5 IU heparin. The plasma was centrifuges, and the cell pellet as incubated on the rolling mixer. The plasma was spiked with LPS and used for batch test I:

1.3. LPS-Spike, Polymyxin-B Solutions, and Batch Test I

LPS: Pseudomonas aeruginosa (L-7018, Sigma Lot Company: 128K4115, −70° C., 10⁻³ g/ml (1 mg/ml)) LPS: E. coli (L-4130, Sigma Lot Company: 110M4086M, −70° C., 10⁻³ g/ml (1 mg/ml))

The LPS was used in the batch with a final concentration of 0.5 ng/ml. The batches were placed in 3-ml pyrogen-free glass vials. In batch test I, various PMB concentrations were placed in double batches and incubated for 60 minutes on an Überkopf shaker at 37° C. (see Table 2).

In batch test 1, PMB concentrations with 0 (no PMB), 10, 100, 250, 500, and 1000 ng/ml were used. For this, sterile PMB solutions (pyrogen-free, autoclaved at 121° C., 90 minutes) were produced with the following concentrations (Table 1):

TABLE 1

| | | PMB |
|---|---|---|
| | PMB [ng/ml] | [ng/ml] im Batch (1:15) |
| PMB-Lösung A | 150 | 10 |
| PMB-Lösung B | 1500 | 100 |
| PMB-Lösung C | 3750 | 250 |
| PMB-Lösung D | 7500 | 500 |
| PMB-Lösung E | 15000 | 1000 |
| NaCl-Lösung | 0 | 0 |

[“Lösung” = solution; “im” = “in the”]

1.4. Endotoxin Analysis

With the aid of a Limulus Amebocyte Lysate test (LAL) from Charles River, the endotoxins were measured in the form of EU/ml.

1.5. Cytokine Batch (Batch Test II)

Plasma spiked with LPS and PMB was tested after batch test I and returned to the cell concentrate obtained from the blood donation in a 1:1 ratio (see Table 2). For the cytokine batch, the samples were brought in at PMB concentrations of 0 (no PMB), 250, 500, and 1000 ng/ml. A sample without LPS and with 1000 ng/ml PMB was used As a control, After incubation times of 4 and 12 hours at 37° C. on the rolling mixer ((5 rpm), samples were taken, centrifuged, and k frozen in 50 μl plasma at −80° C. for the later cytokine quantification. The experimental data for the cytokine batch are listed in Table 2.

TABLE 2

| PMB | | Plasma + 0.5 | | | | | | |
|---|---|---|---|---|---|---|---|---|
| [ng/ml] | PMB-Lsg | ng/ml LPS | Inkubation | LAL | EU/ml | Cytokin-Batch | Probe 4 h | Probe 12 h |
| LPS Pseudomonas aeruginosa | | | | | | | | |
| 0 | 100 μl NaCl | 1400 μl | 60 min | #1 | 0.333 | 1500 μl Zellenkonzentrat + | 250 μl→ 50 μl Plasma −80° C. | 250 μl→ 50 μl |
| 0 | 100 μl NaCl | 1400 μl | 60 min | #2 | 0.229 | 1500 μl LPS-PMB Plasma | | Plasma −80° C. |
| 10 | 100 μl Lsg A | 1400 μl | 60 min | #3 | 0.178 | | | |
| 10 | 100 μl Lsg A | 1400 μl | 60 min | #4 | 0.167 | | | |
| 100 | 100 μl Lsg B | 1400 μl | 60 min | #5 | 0.112 | | | |
| 100 | 100 μl Lsg B | 1400 μl | 60 min | #6 | 0.137 | | | |
| 250 | 100 μl Lsg C | 1400 μl | 60 min | #7 | 0.108 | 1500 μl Zellenkonzentrat + | 250 μl→ 50 μl Plasma −80° C. | 250 μl→ 50 μl |
| 250 | 100 μl Lsg C | 1400 μl | 60 min | #8 | 0.123 | 1500 μl LPS-PMB Plasma | | Plasma −80° C. |
| 500 | 100 μl Lsg D | 1400 μl | 60 min | #9 | 0.091 | 1500 μl Zellenkonzentrat + | 250 μl→ 50 μl Plasma −80° C. | 250 μl→ 50 μl |
| 500 | 100 μl Lsg D | 1400 μl | 60 min | #10 | 0.081 | 1500 μl LPS-PMB Plasma | | Plasma −80° C. |
| 1000 | 100 μl Lsg E | 1400 μl | 60 min | #11 | 0.062 | 1500 μl Zellenkonzentrat + | 250 μl→ 50 μl Plasma −80° C. | 250 μl→ 50 μl |
| 1000 | 100 μl Lsg E | 1400 μl | 60 min | #12 | 0.061 | 1500 μl LPS-PMB Plasma | | Plasma −80° C. |
| LPS E. coli | | | | | | | | |
| 0 | 100 μl NaCl | 1400 μl | 60 min | #13 | 1.8 | 1500 μl Zellenkonzentrat + | 250 μl→ 50 μl Plasma −80° C. | 250 μl→ 50 μl |
| 0 | 100 μl NaCl | 1400 μl | 60 min | #14 | 1.841 | 1500 μl LPS-PMB Plasma | | Plasma −80° C. |
| 10 | 100 μl Lsg A | 1400 μl | 60 min | #15 | 0.77 | | | |
| 10 | 100 μl Lsg A | 1400 μl | 60 min | #16 | 0.871 | | | |
| 100 | 100 μl Lsg B | 1400 μl | 60 min | #17 | 0.379 | | | |
| 100 | 100 μl Lsg B | 1400 μl | 60 min | #18 | 0.382 | | | |
| 250 | 100 μl Lsg C | 1400 μl | 60 min | #19 | 0.281 | 1500 μl Zellenkonzentrat + | 250 μl→ 50 μl Plasma −80° C. | 250 μl→ 50 μl |
| 250 | 100 μl Lsg C | 1400 μl | 60 min | #20 | 0.29 | 1500 μl LPS-PMB Plasma | | Plasma −80° C. |
| 500 | 100 μl Lsg D | 1400 μl | 60 min | #21 | 0.209 | 1500 μl Zellenkonzentrat + | 250 μl→ 50 μl Plasma −80° C. | 250 μl→ 50 μl |
| 500 | 100 μl Lsg D | 1400 μl | 60 min | #22 | 0.209 | 1500 μl LPS-PMB Plasma | | Plasma −80° C. |
| 1000 | 100 μl Lsg E | 1400 μl | 60 min | #23 | 0.154 | 1500 μl Zellenkonzentrat + | 250 μl→ 50 μl Plasma −80° C. | 250 μl→ 50 μl |
| 1000 | 100 μl Lsg E | 1400 μl | 60 min | #24 | 0.16 | 1500 μl LPS-PMB Plasma | | Plasma −80° C. |

“Cytokin” = “Cytokine”
“h” = “hours”
“Inkubation” = “Incubation”
“Lsg” = “solution”
“Probe” = “Sample”
“Zellkonzentrat” = Cell concentrate”

1.6. Results

Endotoxin Batch (Batch Test I):

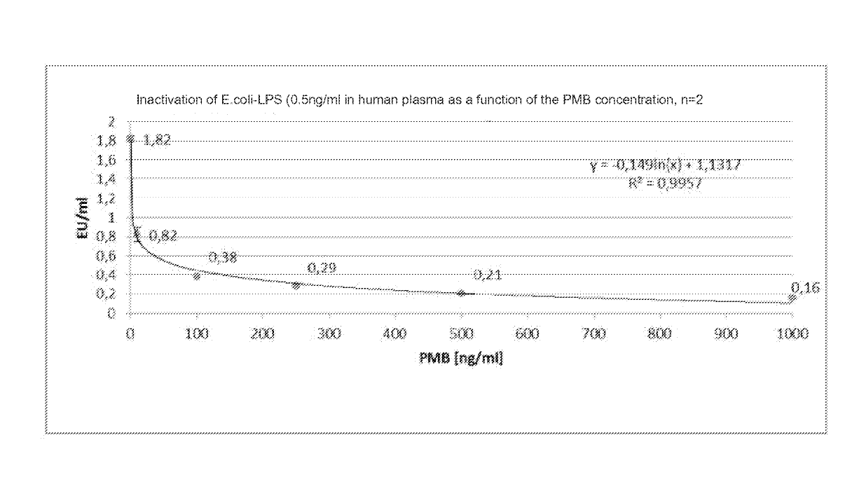

FIG. 1 shows the inhibiting of LPS from E. coli in plasma (original LPS concentration: 0.5 ng/ml) as a function of the PMB concentration (n=2) after an incubation time of 60 minutes. 60 min.

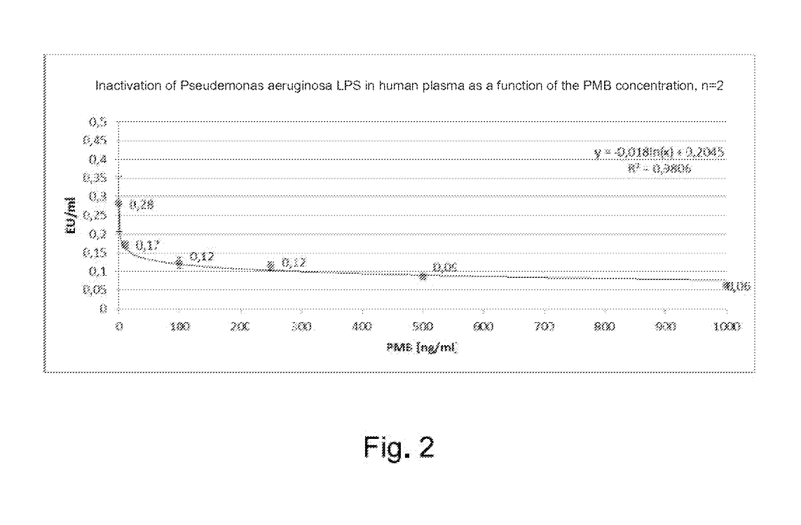

FIG. 2 shows the inhibiting by from Pseudomonas aeruginosa in plasma (original LPS concentration: 0.5 ng/ml) as a function of the PMB concentration (n=2) after an incubation time of 60 minutes.

The results show clearly that already with a very low PBM concentration in the plasma, i.e., in the range from 50 to 300 ng/ml (0.05 to 0.3 μg/ml), a strong inhibition by LPS from E. coli and Pseudomonas aeruginosa takes place, whereby with increasing PMB concentration, the LP inhibition no longer increases significantly. Consequently, very low concentrations of PMB are already sufficient to inhibit LPS (endotoxins) in their activity. At these low concentrations, neuro- and/or nephrotoxic side effects are excluded.

Cytokine Batch (Batch Test II):

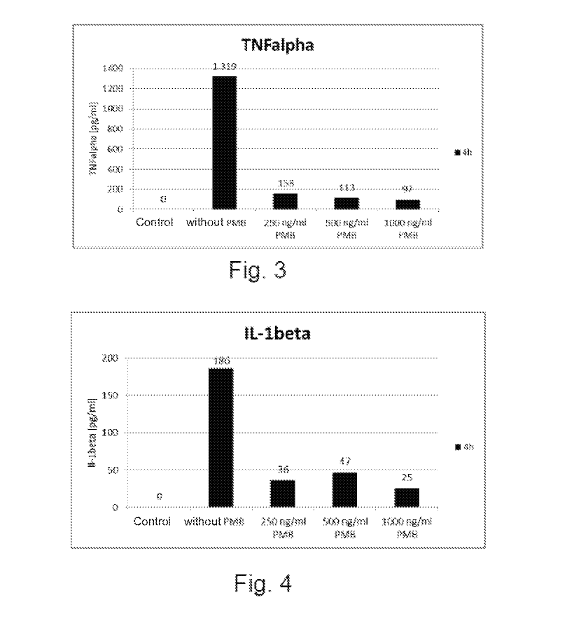

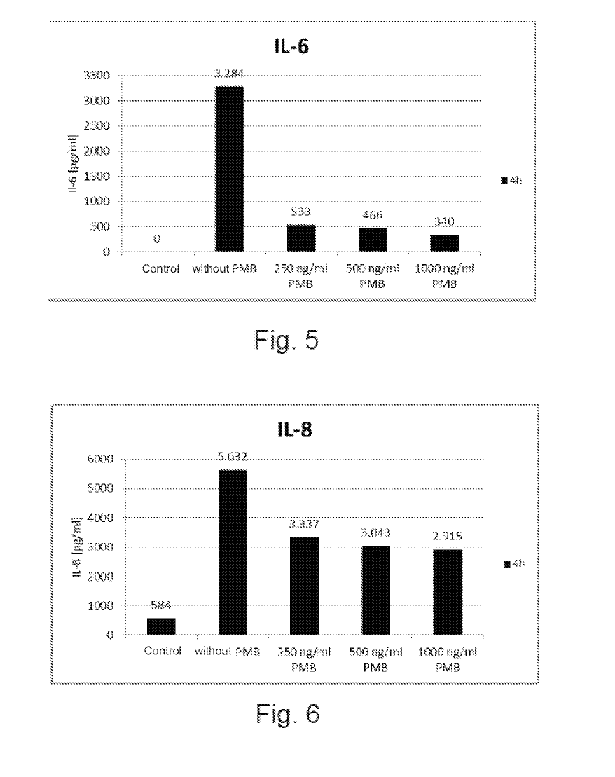

The release of the cytokines TNF-alpha (FIG. 3), IL-1beta (FIG. 4), IL-6 (FIG. 5), and IL-8 (FIG. 6) by the blood cells as a function of the PMB concentration (no PMB, 250 ng/ml, 500 ng/ml, 1000 ng/ml; control with 1000 ng/ml without LPS) in plasma spiked with LPS (E. coli) after 4 hours of incubation is shown in FIGS. 3 through 6. The results from batch test II show clearly that already at very low PMB concentrations, not only a strong inhibition of LPS (see batch test II), but also a strong inhibition of cytokinase release takes place. This is especially pronounced in the inhibition of the key mediator TNF-alpha (FIG. 3).

### 2. EXAMPLE 2

### Dosing Instructions for Polymyxin B with Direct Intravenous for Polymyxin B and Examples of Formulations for Preparation for Parenteral Administration of Polymyxin B (PMB)

2.1. Injection Solutions for Giving a Bolus:

2.1.1. Bolus Administration for a Serum Concentration of 100 ng/ml of Plasma

Assumption: Patient with body weight of 70 kg and 60% of the body weight is a distribution volume for PMB→distribution volume of 42,000 ml.

A serum PMB concentration of 100 ng PMB/ml of plasma is desired→a total of 4.2 mg PMB is needed.

Injection solution for giving a bolus over a time period of 60 minutes: 4.2 mg PMB in 100 ml of physiological saline solution=injection solution ready for administering a bolus over a time period of 60 minutes.

2.1.2. Bolus Administration for a Serum PMB Concentration of 250 ng/ml in Plasma

Assumption: Patient with body weight of 70 kg and 60% of the body weight is a distribution volume for PMB→distribution of volume of 42,000 ml.

A serum PMB concentration of 250 ng PMB/ml of plasma is desired→a total of 10.5 mg PMB is needed.

Injection solution for administering a bolus over a time period of 120 minutes: 10.5 mg PMB in 100 ml physiological saline solution=injection solution ready for administering a bolus over a time period of 120 minutes.

2.2. Infusion Solutions for Maintaining the Serum Concentration):

Assumption: Patient with 70 kg of body weight→Distribution volume for PMB (60% of the body mass) 42,000 ml body fluid with 100 ng PMB/ml→4.2 mg PMB in the distribution volume (see under 2.1.1.).

Infusion solution for a 24-hour infusion with a serum half-life of 6 hours: Assumed half-life of 6 hours for PMB in serum: 2.1 mg PMB per 6 hours or 8.4 mg PMB/day is broken down→8.4 mg PMB in 1 liter physiological saline solution=infusion solution for a 24-hour infusion.

PMB solution for a 24-hour infusion with a serum half-life of 14 hours: Half-life for PMB in serum of 14 hours: 4.2 mg PMB/14 hours or 7.2 mg PMB/day are decomposed→7.2 mg PMB in 1 liter physiological saline solution=infusion solution for a 24-hour infusion.

### 3. EXAMPLE 3

### Dosing Instructions for Polymyxin B (PMB) During Extracorporeal Blood Cleaning (Dialysis and Adsorption Treatment) to Maintain an Already Existing Serum PMB Concentration

The blood-cleaning device according to a known type includes an extracorporeal blood circulation into which the blood of the patient is led and a dialyzer (dialysis filter) is arranged in the extracorporeal blood circulation. It is also assumed in the calculation example that the blood-cleaning device is an adsorption system, for example in the form of an adsorption cartridge, whereby the adsorber system can be connected on the blood side to an extracorporeal blood circulation through a plasma filter (hemoperfusion) or to a plasma filter to the extracorporeal blood circulation through a plasma circulation (plasma or fractionated plasma adsorption.

The following calculation example assumes an already existing serum PMB concentration. This takes place through administering a bolus before the start of the dialysis and adsorption treatment. The injection solutions described under example 2/2.1 are used for this.

To calculate the dosage, the PMB clearance of the patient's body, the dialyzer, and the adsorbing system are taken into account:
Die PMB dialysis clearance (CDial) can be determined experimentally. and it depends on the plasma flow and on the type of dialysis filter used. In this example, this [plasma flow] amounts to 60 ml/min.
The PMB clearance of the adsorber (ads) depends on the adsorbing material used and on the filter flow, or in hemoperfusion, on the blood flow. In the example listed, this amounts to 45 ml/min.
In the example listed, the PMB clearance of the patient was determined from the half-life for PMB or 13.6, and it amounted to 36 ml/min.

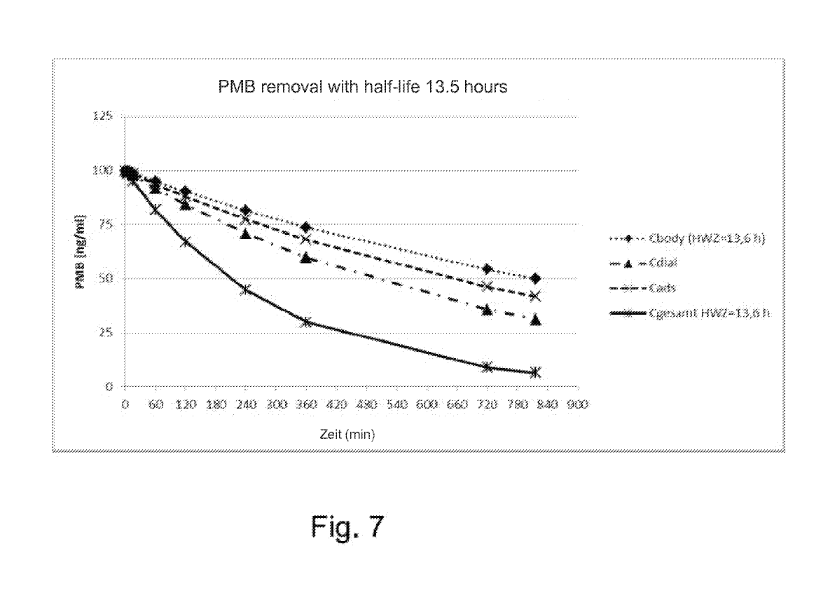

From the individual PMB clearance rates result from adding the total PMB clearance. The resulting assumption for PMB is shown in FIG. 7. The negative increase in PMB removal (Cgesamt) at a particular point in time corresponds to the PMB infusion needed to maintain the serum PMB concentration at the associated time point.

For the example listed, the following infusion rates result:
0.84 mg PMB/hour during treatment with dialysis treatment and adsorption
0.21 mg PMB/hour without dialysis treatment and adsorption

The following quantities of PMB to be infused over 24 hours with 6 hours of extracorporeal treatment result from this:

| | |
|---|---|
| 6 hours of treatment with dialysis and adsorption: | 5.1 mg |
| 18 hours of only PMB infusion (without dialysis and | 3.9 mg |
| adsorption): | |
| Total quantity of PMB infused over 24 hours: | 9.0 mg |

1. A method of treating an endotoxin disorder comprising:
providing an endotoxin-binding lipopeptide selected from the group consisting of polymyxins, polymyxin derivatives, polymyxin analogs, and prodrugs and pharmaceutically acceptable salts thereof for prophylaxis and treatment of diseases and conditions caused by endotoxinemia;
administering a bolus of the lipopeptide parenterally to achieve a serum lipopeptide concentration of 0.01 μg/ml to 0.8 μg/ml; and
maintaining the serum lipopeptide concentration through parenteral administration of the lipopeptide over a treatment time period.

2. The method according to claim 1, whereby the lipopeptide is a polymyxin.

3. The method according to claim 2, whereby the lipopeptide is selected from the group consisting of polymyxin B and colistin.

4. The method according to claim 3, whereby the lipopeptide is polymyxin B.

5. A method of treating an endotoxin disorder comprising:
providing a preparation for parenteral administration for prophylaxis or treatment of diseases and conditions caused by endotoxinemia comprising at least an effective ingredient comprising at least one endotoxin-binding lipopeptide selected from the group consisting of polymyxins, polymyxin derivatives, polymyxin analogs, and prodrugs and pharmaceutically acceptable salts thereof for prophylaxis and treatment of diseases and conditions caused by endotoxinemia, and at least one pharmaceutically acceptable carrier or excipient;
administering a bolus of the lipopeptide preparation parenterally to achieve a serum lipopeptide concentration of 0.01 μg/ml to 0.8 μg/ml; and
maintaining the serum lipopeptide concentration through parenteral administration of the lipopeptide preparation over a treatment time period.

6. The method according to claim 5 wherein the preparation is in the form of an injection preparation or an infusion preparation.

7. The method according to claim 5, whereby the lipopeptide is present in the preparation in a dissolved form selected from a concentration of from 5 mg/l to 200 mg/l for parental administration in a bolus, in a concentration of from 0.04 mg/l to 13 mg/l for parental administration for maintaining the serum lipopeptide concentration, in a concentration of from 0.1 mg/l to 7 mg/l for parental administration for maintaining the serum lipopeptide concentration, and in a concentration of from 0.5 mg/l to 4 mg/l for parental administration for maintaining the serum lipopeptide concentration.

8. The method according to claim 1 wherein the treatment time period for parenteral administration of the bolus is selected from the group of at least 10 minutes, at least 60 minutes, and at least 120 minutes.

9. The method according to claim 1 wherein the serum lipopeptide concentration lies in a range selected from the group of from 0.1 μg/ml to 0.6 μg/ml, from 0.1 μg/ml to 0.4 μg/ml, and from 0.1 μg/ml to 0.25 μg/ml.

10. The method according to claim 1, whereby the serum lipopeptide concentration is maintained through an intravenous administration.

11. The method according to claim 1, wherein the serum lipopeptide concentration is maintained through an infusion into an extracorporeal blood circulation of an extracorporeal perfusion system arranged at a position upstream from a dialyzer.

12. The method according to claim 11, further comprising determining an amount of lipopeptide clearance of the body and an amount of lipopeptide clearance of a dialyzer in administering and maintaining the dosing of the lipopeptide infused into the extracorporeal blood circulation.

13. The method according to claim 12 further comprising determining the lipopeptide clearance of at least one of the a plurality of enrichment devices of the extracorporeal perfusion system.

14. The method according to claim 1, wherein the method is effective for treating an infection selected from the group of Gram-negative bacteria, prophylaxis, systemic inflammatory reaction (SIRS), sepsis, serious sepsis, or septic shock.

15. The method according to claim 1 wherein the method is effective for prophylaxis or treatment of at least one of the following an inflammatory reaction due to an acute liver failure, an acute decompensation in chronic liver failure, a systemic inflammatory reaction (SIRS), sepsis, severe sepsis, or septic shock.

16. The method according to claim 5 wherein the treatment time period for parenteral administration of the bolus is selected from the group of at least 10 minutes, at least 60 minutes, and at least 120 minutes.

17. The method according to claim 5 wherein the serum lipopeptide concentration lies in a range selected from the group of from 0.1 μg/ml to 0.6 μg/ml, from 0.1 μg/ml to 0.4 μg/ml, and from 0.1 μg/ml to 0.25 μg/ml.

18. The method according to claim 5, whereby the serum lipopeptide concentration is maintained through a technique selected from the group of intravenous administration, infusion into an extracorporeal blood circulation of an extracorporeal perfusion system, and infusion into an extracorporeal blood circulation of the blood of a patient arranged at a position upstream from a dialyzer.

19. The method according to claim 5, wherein the method is effective for treating an infection selected from the group of Gram-negative bacteria, prophylaxis, systemic inflammatory reaction (SIRS), sepsis, serious sepsis, or septic shock.

20. The method according to claim 5 wherein the method is effective for prophylaxis or treatment of at least one of the following an inflammatory reaction due to an acute liver failure, an acute decompensation in chronic liver failure, a systemic inflammatory reaction (SIRS), sepsis, severe sepsis, or septic shock.
